(19) United States
(12) Patent Application Publication
(10) Pub. No.: US 2009/0234193 A1
(43) Pub. Date: Sep. 17, 2009

(54) APPARATUS FOR KEEPING CLEAN A DISTAL SCOPE END OF A MEDICAL VIEWING SCOPE

(75) Inventors: William B. Weisenburgh, II, Maineville, OH (US); Carl J. Shurtleff, Mason, OH (US); Christopher J. Hess, Cincinnati, OH (US); Michael A. Murray, Bellevue, KY (US); James W. Voegele, Cincinnati, OH (US); Darrel M. Powell, Cincinnati, OH (US)

Correspondence Address: THOMPSON HINE L.L.P.;Intellectual Property Group, P.O. BOX 8801, DAYTON, OH, 45401-8801, US

(73) Assignee: ETHICON ENDO-SURGERY, INC., Cincinnati, OH (US)

(21) Appl. No.: 12/047,474

(22) Filed: Mar. 13, 2008

(51) Int. Cl. A61B 1/12

(52) U.S. Cl. 600/157

(57) ABSTRACT

A first expression of apparatus for keeping clean a medical viewing scope's distal scope end includes a tube, an annular sheath, and a handpiece. The tube has a proximal end fluidly connectable to irrigation fluid and has a distal end fluidly connected to the handpiece. The sheath is surroundingly attachable to the scope and includes a lumen between the sheath's inside and outside diameters. The lumen has a substantially constant cross-sectional flow area which is substantially equal in area to the tube's cross-sectional flow area. The handpiece is in fluid communication with the proximal lumen end. The distal scope end is positioned proximate the attached sheath's distal lumen end. A second expression of the apparatus has the lumen with a substantially crescent shape without any limitation on its cross-sectional flow area. In an alternate embodiment, the lumen substantially continuously varies in cross-sectional flow area and/or irrigation flow path direction.

### CROSS REFERENCE TO RELATED PATENT APPLICATIONS

This patent application incorporates by reference U.S. patent application Ser. No. 11/542,060 filed Oct. 3, 2006.

### FIELD OF THE INVENTION

The present invention is related generally to medical equipment, and more particularly to apparatus for keeping clean a distal scope end of a medical viewing scope.

### BACKGROUND OF THE INVENTION

During some conventional laparoscopic procedures, first and second trocars are used to create two openings in the patient's abdomen. A rigid laparoscope is inserted through the first trocar to visualize patient tissue. A treating medical instrument is inserted through the second trocar to treat the patient tissue being visualized with the laparoscope. Bodily fluid dispersion and floating debris have a tendency to accumulate on the scope lens of the inserted laparoscope degrading the clarity of the view. Thus, at times during the laparoscopic procedure the laparoscope must be withdrawn from the first trocar and the scope lens wiped to remove the accumulated material which caused the blurred viewing. The removal of the laparoscope is inconvenient and causes delays in the laparoscopic procedure. Upon reinsertion of the laparoscope, it is necessary for the physician to take additional time to maneuver the scope to reacquire the patient tissue of interest.

### SUMMARY

A first expression of a first embodiment of the invention is for apparatus for keeping clean a distal scope end of a medical viewing scope. The apparatus includes a first tube, an annular sheath, and a handpiece. The first tube has a first proximal tube end fluidly connectable to an irrigation fluid source, has a first distal tube end, and has a substantially constant first cross-sectional flow area. The sheath is surroundingly attachable to the scope, wherein the sheath includes a tubular wall having inside and outside diameters and containing a lumen between the inside and outside diameters. The lumen has proximal and distal lumen ends, wherein the lumen has a substantially constant cross-sectional flow area which is substantially equal in area to the first cross-sectional flow area. The distal scope end is positioned proximate the distal lumen end of the attached sheath. The handpiece is mounted to the sheath, is fluidly connected to the first distal tube end, and is in fluid communication with the proximal lumen end. The handpiece is adapted to have a user-selectable first internal flow configuration preventing fluid communication between the first distal tube end and the proximal lumen end and a user-selectable second internal flow configuration allowing fluid communication between the first distal tube end and the proximal lumen end.

A second expression of a first embodiment of the invention is for apparatus for keeping clean a distal scope end of a medical viewing scope. The apparatus includes first and second tubes, an annular sheath, and a handpiece. The first tube has a first proximal tube end fluidly connectable to an irrigation fluid source and has a first distal tube end. The second tube has a second proximal tube end fluidly connectable to a vacuum source and has a second distal tube end. The sheath is surroundingly attachable to the scope, wherein the sheath includes a tubular wall having inside and outside diameters and containing a lumen between the inside and outside diameters. The lumen has proximal and distal lumen ends, wherein the lumen has a cross-sectional flow area which has a substantially crescent shape. The distal scope end is positioned proximate the distal lumen end of the attached sheath. The handpiece is mounted to the sheath, is fluidly connected to the first and second distal tube ends, and is in fluid communication with the proximal lumen end. The handpiece is adapted to have a user-selectable first internal flow configuration preventing fluid communication between the first distal tube end and the proximal lumen end and between the second distal tube end and the proximal lumen end, a user-selectable second internal flow configuration allowing fluid communication between the first distal tube end and the proximal lumen end but not between the second distal tube end and the proximal lumen end, and a user-selectable third internal flow configuration allowing fluid communication between the second distal tube end and the proximal lumen end but not between the first distal tube end and the proximal lumen end.

A third expression of a first embodiment of the invention is for apparatus for keeping clean a distal scope end of a medical viewing scope. The apparatus includes first and second tubes, an annular sheath, and a handpiece. The first tube has a first proximal tube end fluidly connectable to an irrigation fluid source, has a first distal tube end, and has a substantially constant first cross-sectional flow area. The second tube has a second proximal tube end fluidly connectable to a vacuum source and has a second distal tube end. The sheath has a central longitudinal axis, is surroundingly attachable to the scope, and is insertable into a patient. The sheath includes a tubular wall having inside and outside diameters and containing a lumen between the inside and outside diameters. The lumen has proximal and distal lumen ends, wherein the lumen has a substantially constant cross-sectional flow area which is substantially equal in area to the first cross-sectional flow area of the first tube. The sheath includes a distal sheath end portion defining a manifold. The manifold has an annular fluid passageway which has a volume and which is in fluid communication with the distal lumen end. The manifold has a plurality of spaced apart nozzle passageways which together have a total volume, which are in fluid communication with the annular fluid passageway, and which point proximal of the annular fluid passageway. The volume of the annular fluid passageway is greater than the total volume of the nozzle passageways. The distal scope end is positioned proximate the nozzle passageways of the attached sheath. The handpiece is mounted to the sheath, is fluidly connected to the first and second distal tube ends, and is in fluid communication with the proximal lumen end. The handpiece is adapted to have a user-selectable first internal flow configuration preventing fluid communication between the first distal tube end and the proximal lumen end and between the second distal tube end and the proximal lumen end, a user-selectable second internal flow configuration allowing fluid communication between the first distal tube end and the proximal lumen end but not between the second distal tube end and the proximal lumen end, and a user-selectable third internal flow configuration allowing fluid communication between the second distal tube end and the proximal lumen end but not between the first distal tube end and the proximal lumen end.

A first expression of a second embodiment of the invention is for apparatus for keeping clean a distal scope end of a medical viewing scope. The apparatus includes an annular sheath surroundingly attachable to the scope. The sheath includes a tubular wall having inside and outside diameters and containing a lumen between the inside and outside diameters. The lumen has proximal and distal lumen ends. The lumen substantially continuously varies in at least one of cross-sectional flow area and irrigation flow path direction. The proximal lumen end is fluidly connectable to at least one of an irrigation fluid source and a vacuum source. The distal scope end is positioned proximate the distal lumen end of the attached sheath.

Several benefits and advantages are obtained from one or more of the expressions of embodiments of the invention which provide for keeping clean a distal scope end of a medical viewing scope while the scope remains inserted in a patient. In one example, not removing the scope for cleaning and not reinserting the cleaned scope reduces the time for a laparoscopic procedure. In the same or a different example, not removing the scope for cleaning and not reinserting the cleaned scope keeps the inserted scope aligned with the patient tissue of interest during cleaning so that the physician does not have to take additional time to maneuver the scope to reacquire the patient tissue of interest. In one example of the first and third expressions of the first embodiment, the substantially equal areas reduce flow losses and provide faster response times for irrigation fluid to exit the lumen of the sheath to clean the distal scope end or to clean a magnifying or non-magnifying optional lens (transparent shield) of the sheath which protects the distal scope end.

### BRIEF DESCRIPTION OF THE FIGURES

FIG. 1 is a schematic top view of a first embodiment of the invention including first and second tubes, an annular sheath, a bellows shown in a fully-extended position, and a handpiece;

FIG. 2 is a schematic side view of the embodiment of FIG. 1;

FIG. 3 is a schematic side view of the embodiment of FIG. 1 together with an irrigation fluid source fluidly connected to the first tube, a vacuum source fluidly connected to the second tube, and a medical viewing scope in the form of a laparoscope having a housing and an insertion tube extending from the housing, wherein the insertion tube has been inserted into the sheath, and wherein other components of the laparoscope have been omitted for clarity;

FIG. 4 is a cross-sectional view of the first tube of FIG. 1, taken along arrows 4-4 of FIG. 1, showing the cross-sectional flow area of the first tube;

FIG. 5 is a cross-sectional view of the second tube of FIG. 1, taken along arrows 5-5 of FIG. 1, showing the cross-sectional flow area of the second tube;

FIG. 6 is a cross-sectional view of the sheath of FIG. 1, taken along arrows 6-6 of FIG. 1, showing the cross-sectional flow area of the lumen of the sheath;

FIG. 7A is a cross-sectional view of the sheath and the distal end portion of the handpiece of FIG. 2, taken along lines 7A-7A of FIG. 2, showing a fluid connection of the distal handpiece passageway portion with the proximal lumen end and showing the manifold defined by the distal sheath end portion, wherein the manifold has an annular fluid passageway in fluid communication with the distal lumen end and has a plurality of spaced apart nozzle passageways in fluid communication with the annular fluid passageway;

FIG. 7B is a cross-sectional view of the sheath of FIG. 7A taken along lines 7B-7B of FIG. 7A;

FIG. 8 is a view, as in FIG. 7A, but also including the scope of FIG. 3 showing the insertion tube of the scope inserted in the sheath;

FIG. 9 is a view, as in FIG. 8, but with the sheath inserted into a trocar which has been inserted into a patient;

FIG. 10 is a diagrammatic view of the handpiece and a distal portion of the first and second tubes of FIG. 1, showing the distal handpiece passageway portion seen in FIG. 7A and illustrating with flow arrows the first internal flow configuration of the handpiece;

FIG. 11 is a diagrammatic view, as in FIG. 10, but illustrating with flow arrows the second internal flow configuration of the handpiece;

FIG. 12 is a diagrammatic view, as in FIG. 10, but illustrating with flow arrows the third internal flow configuration of the handpiece;

FIG. 13 is a cross-sectional view of a first alternate embodiment of the sheath of FIG. 1, wherein the manifold is rotatable and is longitudinally extendable and retractable;

FIG. 14 is an enlarged cross-sectional view of the bellows, the handpiece, the sheath, and the scope of FIG. 3, taken along lines 14-14 of FIG. 3, showing the position of the bellows for a shorter scope;

FIG. 15 is a view, as in FIG. 14, but showing the position of the bellows for a longer scope;

FIG. 16 is a view of an alternate embodiment of the first tube with a fitting which has threadably received a container containing an anti-fogging liquid;

FIG. 17 is a cross-sectional view of the sheath and the distal end portion of the handpiece of FIG. 7A taken along lines 17-17 of FIG. 7A showing the lumen, wherein the lumen has a substantially straight flow path and, from FIG. 6, has a substantially constant cross-sectional flow area which has a substantially crescent shape;

FIG. 18 is a view, as in FIG. 17, but of a second alternate embodiment of the sheath of FIG. 1 showing a substantially helical flow path where the lumen has a substantially constant cross-sectional flow area which has a substantially crescent shape;

FIG. 19 is a cross-sectional view of the sheath of FIG. 18 taken along lines 19-19 of FIG. 18 showing the substantially crescent shape of the cross-sectional flow area of the lumen of the sheath;

FIG. 20 is a side elevational cross-sectional view of portion of a first alternate embodiment of the handpiece of FIG. 1 showing a first valve having a first button adapted to pump irrigation fluid into the proximal lumen end of the lumen of the sheath;

FIG. 21 is a side elevational cross-sectional view of a portion of a second alternate embodiment of the handpiece of FIG. 1 showing a second valve having a second button adapted to provide suction to the lumen proximal end of the lumen of the sheath;

FIG. 22 is a side elevational cross-sectional view of a portion of a third alternate embodiment of the handpiece of FIG. 1 showing a single valve having a button adapted to provide irrigation fluid into the proximal lumen end of the lumen of the sheath when partially depressed and adapted to provide suction to the lumen proximal end of the lumen of the sheath when completely depressed;

FIG. 23 is a view, as in FIG. 17, but of a third alternate embodiment of the sheath of FIG. 1 showing a substantially helical flow path where the lumen has a substantially constant cross-sectional flow area which has a substantially circular shape;

FIG. 24 is a cross-sectional view of the sheath of FIG. 23 taken along lines 24-24 of FIG. 23 showing the substantially circular shape of the cross-sectional flow area of the lumen of the sheath;

FIG. 25 is a view, as in FIG. 17, but of a fourth alternate embodiment of the sheath of FIG. 1 showing a substantially straight flow path where the lumen has a tapered cross-sectional flow area which has a substantially crescent shape; and

FIG. 26 is a cross-sectional view of the sheath of FIG. 25 taken along lines 26-26 of FIG. 25 showing the substantially crescent shape of the cross-sectional flow area of the lumen of the sheath.

### DETAILED DESCRIPTION

Before explaining the several embodiments of the present invention in detail, it should be noted that each embodiment is not limited in its application or use to the details of construction and arrangement of parts and steps illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is further understood that any one or more of the following-described expressions, embodiments, examples, etc. can be combined with any one or more of the other following-described expressions, embodiments, examples, etc.

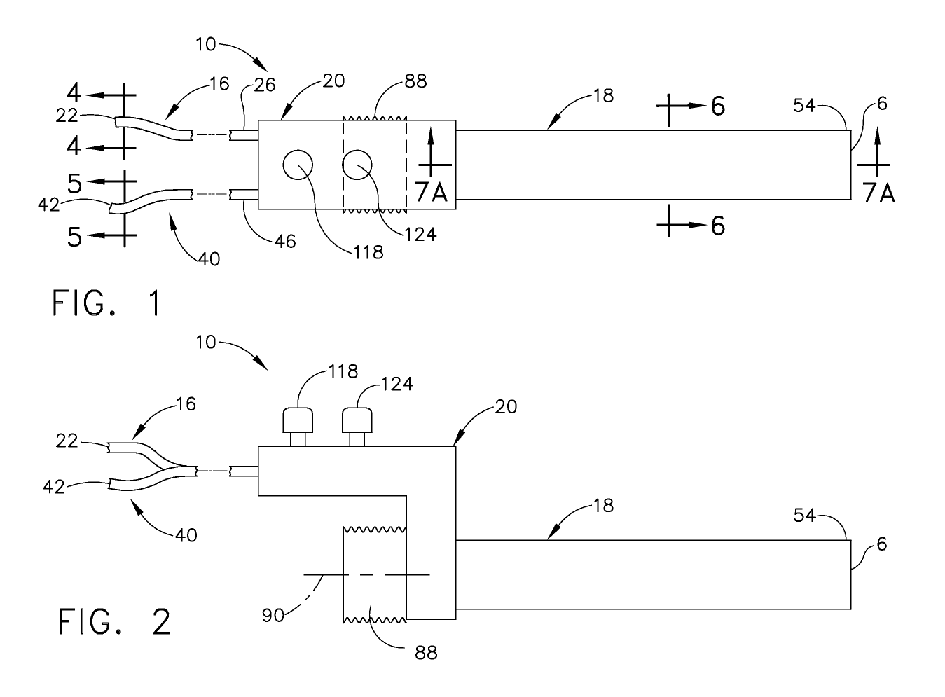

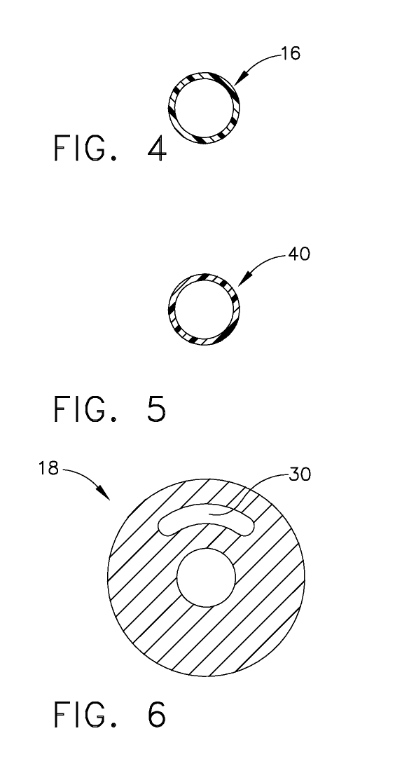

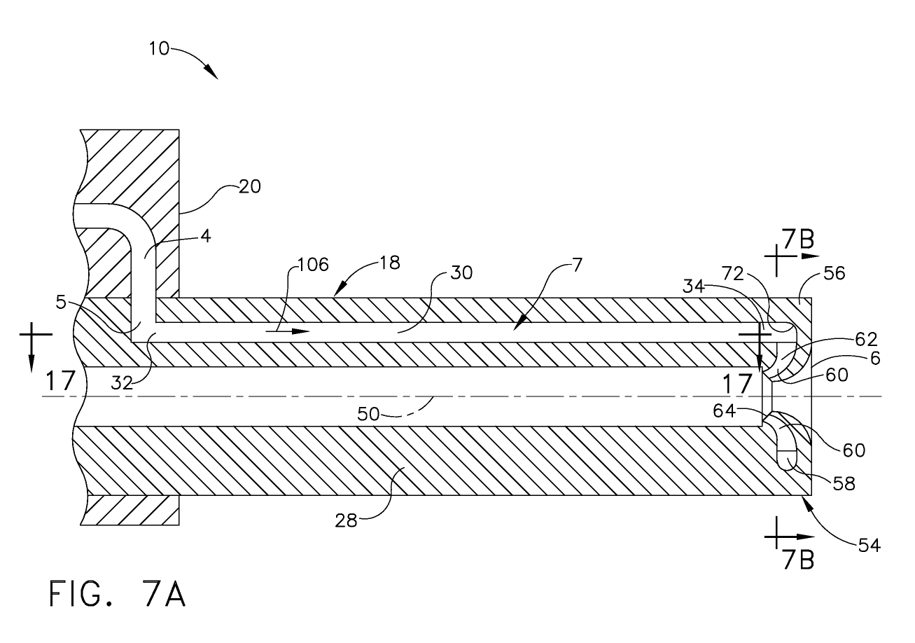

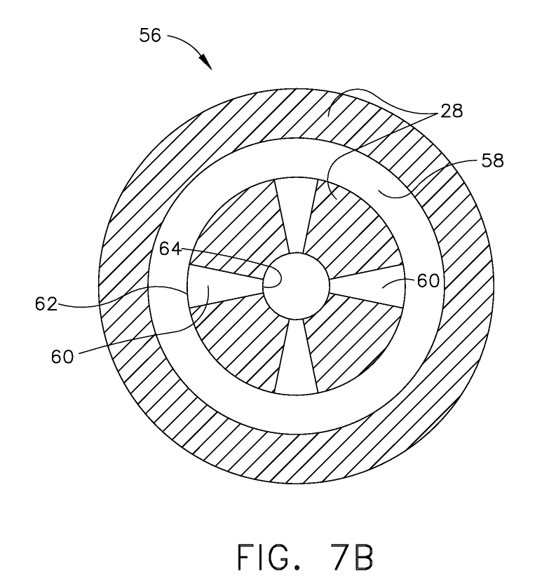

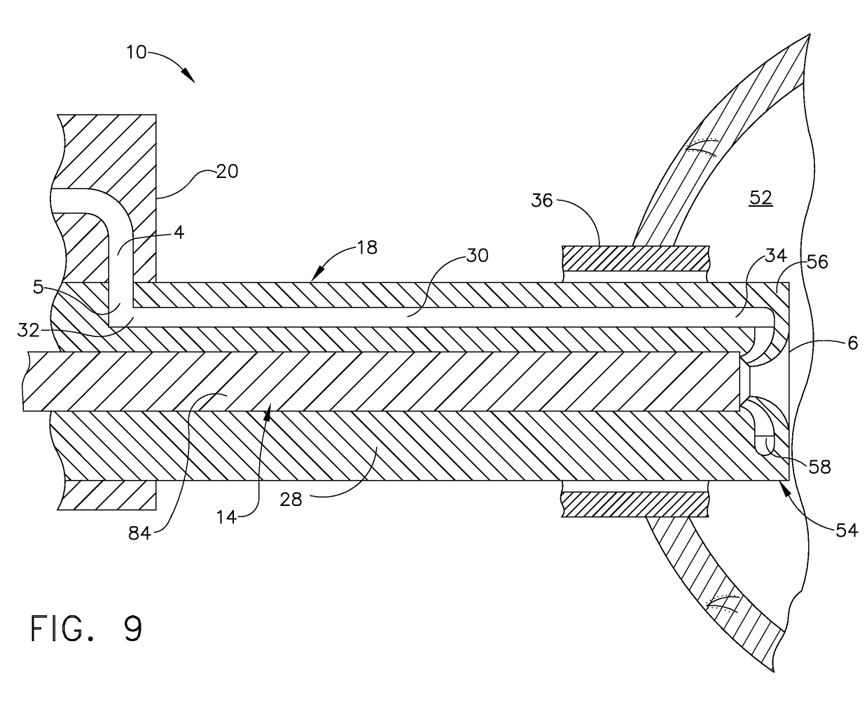

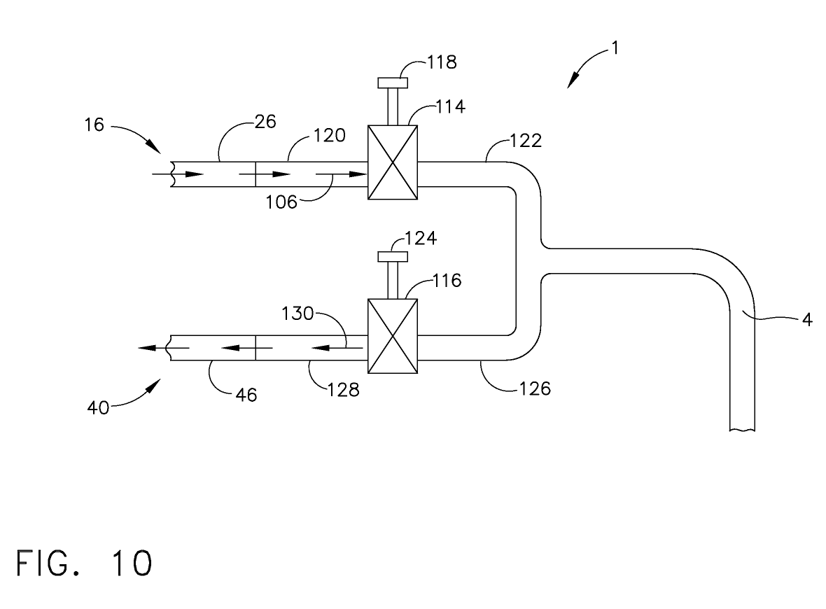

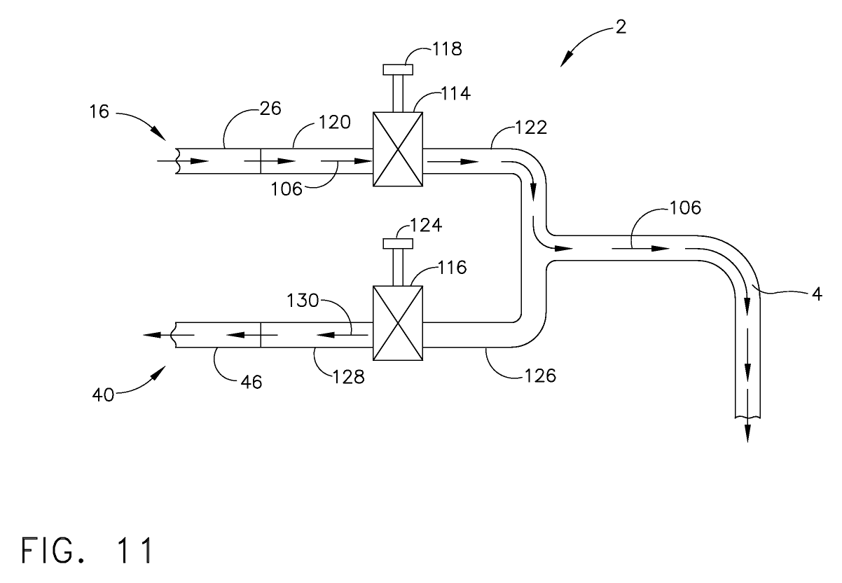

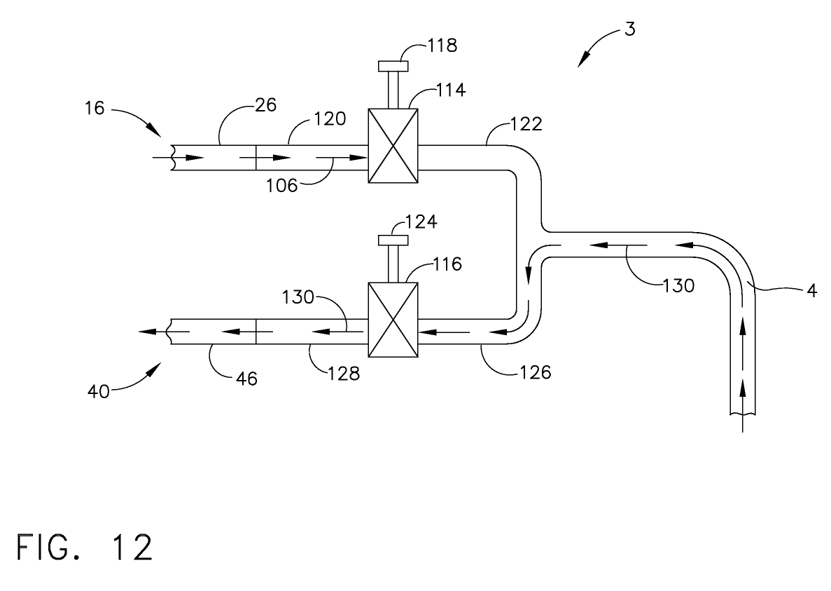

A first embodiment of the invention is shown in FIGS. 1-12, 14-15, and 17. A first expression of the first embodiment, as best seen in FIGS. 1-3, is for apparatus 10 for keeping clean a distal scope end 12 of a medical viewing scope 14. The apparatus 10 includes a first tube 16, an annular sheath 18, and a handpiece 20. The first tube 16 has a first proximal tube end 22 fluidly connectable to an irrigation fluid source 24 and has a first distal tube end 26. As best seen in FIGS. 4-9, the first tube 16 has a substantially constant first cross-sectional flow area. The sheath 18 is surroundingly attachable to the scope 14, wherein the sheath 18 includes a tubular wall 28 having inside and outside diameters and containing a lumen 30 between the inside and outside diameters. The lumen 30 has proximal and distal lumen ends 32 and 34, wherein the lumen 30 has a substantially constant cross-sectional flow area which is substantially equal in area to the first cross-sectional flow area. The distal scope end 12 is disposed proximate the distal lumen end 34 of the attached sheath 18. As best seen in FIGS. 1-3, the handpiece 20 is mounted to the sheath 18, is fluidly connected to the first distal tube end 26, and, as best seen in FIGS. 7-9, is in fluid communication with the proximal lumen end 32. The handpiece 20 is adapted to have, as best seen in FIGS. 9 and 10, a user-selectable first internal flow configuration 1 preventing fluid communication between the first distal tube end 26 and the proximal lumen end 32, and to have, as best seen in FIGS. 9 and 11, a user-selectable second internal flow configuration 2 allowing fluid communication between the first distal tube end 26 and the proximal lumen end 32. It is noted that “fluid” includes, without limitation, gas or gasses (e.g., pressurized air) and/or liquid or liquids.

In one example of the first expression of the first embodiment, the fluid communication between the first distal tube end 26 and the proximal lumen end 32 includes the handpiece 20 having a distal handpiece passageway portion 4 which exits the handpiece 20 and includes the sheath 18 having a proximal sheath passageway portion 5 which enters the sheath 18. In this example, the distal handpiece passageway portion 4 is directly fluidly connected to the proximal sheath passageway portion 5, and the proximal sheath passageway portion 5 is directly fluidly connected to the proximal lumen end 32.

It is noted that “keeping clean a distal scope end 12” includes cleaning at least a portion (such as a scope lens if so equipped) of the distal scope end 12 to improve scope clarity (such as scope lens clarity if so equipped), and includes cleaning at least a portion of a sheath lens (if the sheath is so equipped with a sheath lens adapted to protect the distal scope end) to improve scope clarity. It is also noted describing the first tube 16 as having a substantially constant cross-sectional flow area means the cross-sectional flow area is substantially constant from proximate the first proximal tube end 22 to proximate the first distal tube end 26. It is further noted that describing the lumen 30 as having a substantially constant cross-sectional flow area means the cross-sectional flow area is substantially constant from proximate the handpiece 20 to proximate the distal sheath end 6.

In one sheath-to-scope attachment technique, the scope 14 is slidingly insertable into the proximal sheath end portion near the handpiece 20, the distal scope end 12 has an outside diameter, and, although not shown in the figures, the inside diameter of the tubular wall 28 near the distal sheath end 6 is less than the outside diameter of the distal scope end 12. In one variation, the distal scope end 12 makes a press fit with the sheath 18 near the distal sheath end 6. In one modification, the inside diameter of the tubular wall 24 has a constant taper. Other attachments, not shown, of the sheath 18 to the scope 14 include, without limitation, an elastomeric sheath, a compression fitting, and an elastomeric O-ring attached to the sheath proximate the distal sheath end 6 and adapted to attachingly engage an advancing scope 14 which has been inserted into the proximal sheath end near the handpiece 20.

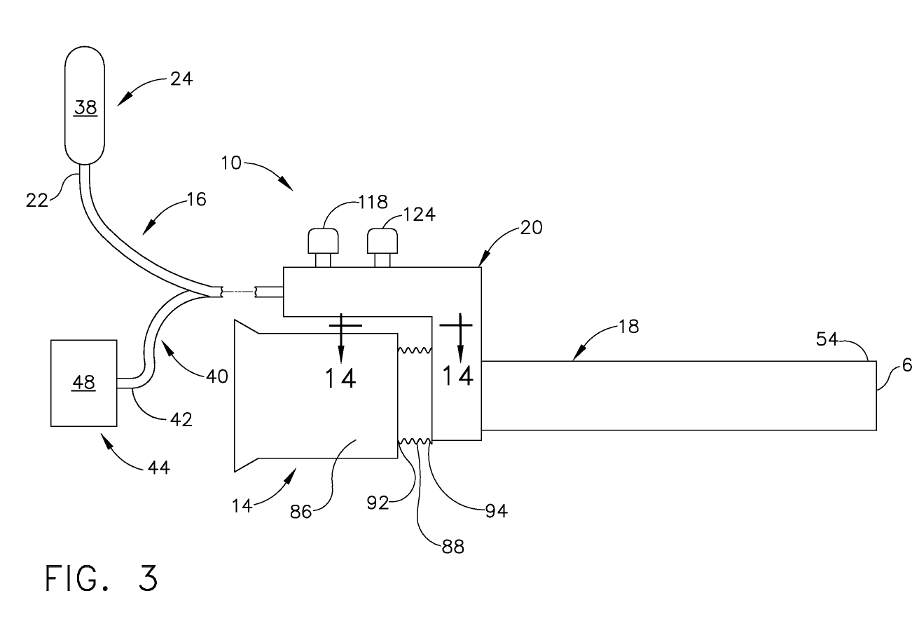

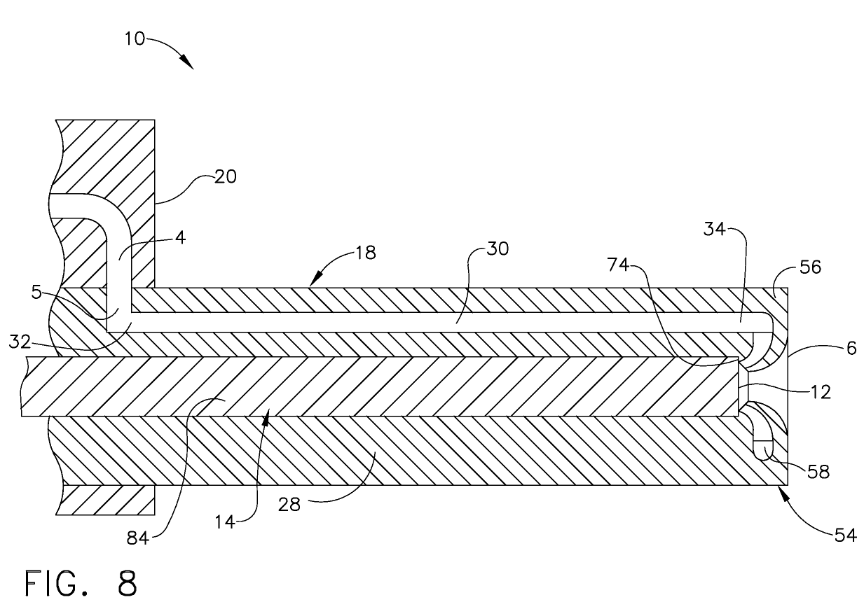

In one enablement of the first expression of the first embodiment, as best seen in FIGS. 8 and 9, the distal scope end 12 is in fluid communication with the distal lumen end 34 of the attached sheath 18. In the same or a different enablement, as best seen in FIG. 4, the first cross-sectional flow area of the first tube 16 has a substantially circular shape, and, as best seen in FIG. 6, the cross-sectional flow area of the lumen 30 of the sheath 18 has a substantially crescent shape. In one variation, as best seen in FIG. 9, the scope 14 is a laparoscope, and the sheath 18 is substantially rigid and is insertable into a trocar 36. Other types of scopes, not shown, include, without limitation, endoscopes (including gastroscopes and colonoscopes). It is noted that scopes include, without limitation, those scopes with video cameras which display an image on a monitor and those scopes having eyepieces for viewing by a physician. In one modification, as best seen in FIG. 3, the irrigation fluid source 24 is an operating-room saline bag 38, and the first proximal tube end 22 is fluidly connected to the saline bag 38.

A second expression of the first embodiment, as best seen in FIGS. 1-3, is for apparatus 10 for keeping clean a distal scope end 12 of a medical viewing scope 14. The apparatus 10 includes first and second tubes 16 and 40, an annular sheath 18, and a handpiece 20. The first tube 16 has a first proximal tube end 22 fluidly connectable to an irrigation fluid source 24 and has a first distal tube end 26. The second tube 40 has a second proximal tube end 42 fluidly connectable to a vacuum source 44 and has a second distal tube end 46. The sheath 18 is surroundingly attachable to the scope 14, wherein the sheath 18 includes a tubular wall 28 having inside and outside diameters and containing a lumen 30 between the inside and outside diameters. The lumen 30 has proximal and distal lumen ends 32 and 34, wherein the lumen 30 has a cross-sectional flow area which has a substantially crescent shape. The distal scope end 12 is disposed proximate the distal lumen end 34 of the attached sheath 18. As best seen in FIGS. 1-3, the handpiece 20 is mounted to the sheath 18, is fluidly connected to the first and second distal tube ends 26 and 46, and, as best seen in FIGS. 7-9, is in fluid communication with the proximal lumen end 32.

In the second expression of the first embodiment, the handpiece 20 is adapted to have, as best seen in FIGS. 9 and 10, a user-selectable first internal flow configuration 1 preventing fluid communication between the first distal tube end 26 and the proximal lumen end 32 and between the second distal tube end 46 and the proximal lumen end 32, to have, as best seen in FIGS. 9 and 11, a user-selectable second internal flow configuration 2 allowing fluid communication between the first distal tube end 26 and the proximal lumen end 32 but not between the second distal tube end 46 and the proximal lumen end 32, and to have, as best seen in FIGS. 9 and 12, a user-selectable third internal flow configuration 3 allowing fluid communication between the second distal tube end 46 and the proximal lumen end 32 but not between the first distal tube end 26 and the proximal lumen end 32.

It is noted that describing the second tube 40 as having a substantially constant cross-sectional flow area means the cross-sectional flow area is substantially constant from proximate the second proximal tube end 42 to proximate the second distal tube end 46. It is also noted that the term “vacuum” includes partial vacuum and includes aspiration. It is further noted that the term “vacuum” is relative to the pressure proximate the distal scope end 12 and that, in one example, the vacuum source may be ambient room air when the distal scope end 12 is exposed to a higher pressure within, for example, the insufflated abdomen of a patient 52.

In one enablement of the second expression of the first embodiment, as best seen in FIGS. 8 and 9, the distal scope end 12 is in fluid communication with the distal lumen end 34 of the attached sheath 18. In the same or a different enablement, as best seen in FIGS. 4-6, the first tube 16 has a substantially constant first cross-sectional flow area, the second tube 40 has a substantially constant second cross-sectional flow area which is substantially equal in area to the first cross-sectional flow area of the first tube 16, the lumen 30 of the sheath 18 has a substantially constant cross-sectional flow area, the first and second cross-sectional flow areas each have a substantially circular shape, and the cross-sectional flow area of the lumen 30 of the sheath 18 is substantially equal in area to the first cross-sectional flow area of the first tube 16. In one variation, as best seen in FIG. 9, the scope 14 is a laparoscope, and the sheath 18 is substantially rigid and is insertable into a trocar 36. In one example, the first and second tubes 16 and 40 are each ten feet of flexible tubing. In one modification, as best seen in FIG. 3, the irrigation fluid source 24 is an operating-room saline bag 38, the first proximal tube end 22 is fluidly connected to the saline bag 38, the vacuum source 44 is an operating-room suction canister 48, and the second proximal tube end 42 is fluidly connected to the suction canister 48.

A third expression of the first embodiment, as best seen in FIGS. 1-3, is for apparatus 10 for keeping clean a distal scope end 12 of a medical viewing scope 14. The apparatus 10 includes first and second tubes 16 and 40, an annular sheath 18, and a handpiece 20. The first tube 16 has a first proximal tube end 22 fluidly connectable to an irrigation fluid source 24, has a first distal tube end 26, and has a substantially constant first cross-sectional flow area. The second tube 40 has a second proximal tube end 42 fluidly connectable to a vacuum source 44 and has a second distal tube end 46. The sheath 18 has a central longitudinal axis 50, is surroundingly attachable to the scope 14, and is insertable into a patient 52. The sheath 18 includes a tubular wall 28 having inside and outside diameters and containing a lumen 30 between the inside and outside diameters. The lumen 30 has proximal and distal lumen ends 32 and 34, wherein the lumen 30 has a substantially constant cross-sectional flow area which is substantially equal to the first cross-sectional flow area of the first tube 16.

In the third expression of the first embodiment, the sheath 18 includes, as best seen in FIGS. 7-9, a distal sheath end portion 54 defining a manifold 56. The manifold 56 has an annular fluid passageway 58 which has a volume and which is in fluid communication with the distal lumen end 34. The manifold 56 has a plurality of spaced apart nozzle passageways 60 (two are shown in FIG. 7A and four are shown in FIG. 7B) which together have a total volume, which are in fluid communication with the annular fluid passageway 58, and which point proximal of the annular fluid passageway 58. The volume of the annular fluid passageway 58 is greater than the total volume of the nozzle passageways 60. The distal scope end 12 is disposed proximate the nozzle passageways 60 of the attached sheath 18. As best seen in FIGS. 1-3, the handpiece 20 is mounted to the sheath 18, is fluidly connected to the first and second distal tube ends 26 and 46, and, as best seen in FIGS. 7-9, is in fluid communication with the proximal lumen end 32.

In the third expression of the first embodiment, the handpiece 20 is adapted to have, as best seen in FIGS. 9 and 10, a user-selectable first internal flow configuration 1 preventing fluid communication between the first distal tube end 26 and the proximal lumen end 32 and between the second distal tube end 46 and the proximal lumen end 32, to have, as best seen in FIGS. 9 and 11, a user-selectable second internal flow configuration 2 allowing fluid communication between the first distal tube end 26 and the proximal lumen end 32 but not between the second distal tube end 46 and the proximal lumen end 32, and to have, as best seen in FIGS. 9 and 12, a user-selectable third internal flow configuration 3 allowing fluid communication between the second distal tube end 46 and the proximal lumen end 32 but not between the first distal tube end 26 and the proximal lumen end 32.

In one example of the third expression of the embodiment, as best seen in FIG. 7A, the sheath 66 includes a sheath passageway 7 consisting essentially of the proximal sheath passageway portion 5, the lumen 30, the annular fluid passageway 58 of the manifold 56, and the nozzle passageways 60 of the manifold 56.

In one enablement of the third expression of the first embodiment, as best seen in FIGS. 8 and 9, the distal scope end 12 is in fluid communication with the nozzle passageways 60. In the same or a different enablement, as best seen in FIGS. 4-6, the second tube 40 has a substantially constant second cross-sectional flow area which is substantially equal in area to the first cross-sectional flow area of the first tube 16, the first and second cross-sectional flow areas each have a substantially circular shape, and the cross-sectional flow area of the lumen 30 of the sheath 18 has a substantially crescent shape. In one example, as best seen in FIG. 6, the crescent shape is substantially equal in shape to an end view of substantially ninety degrees of a circularly annular right cylinder. In one variation, as best seen in FIG. 9, the scope 14 is a laparoscope, and the sheath 18 is substantially rigid and is insertable into a trocar 36. In one modification, as best seen in FIG. 3, the irrigation fluid source 24 is an operating-room saline bag 38, the first proximal tube end 22 is fluidly connected to the saline bag 38, the vacuum source 44 is an operating-room suction canister 48, and the second proximal tube end 42 is fluidly connected to the suction canister 48.

In one implementation of the third expression of the first embodiment, as best seen in FIGS. 7A and 7B, each nozzle passageway 60 has a proximal nozzle passageway end 62 and a distal nozzle passageway end 64, and each nozzle passageway 60 is tapered from the corresponding proximal nozzle passageway end 62 to the corresponding distal nozzle passageway end 64. In the same or a different implementation, as best seen in FIG. 7A, the distal nozzle passageway end 64 of the attached sheath 18 has a portion abutting the distal scope end 12 and a portion spaced apart from the distal scope end 12. In one variation, each nozzle passageway 60 is aligned to intersect the central longitudinal axis 50 of the sheath 18 at substantially the same point.

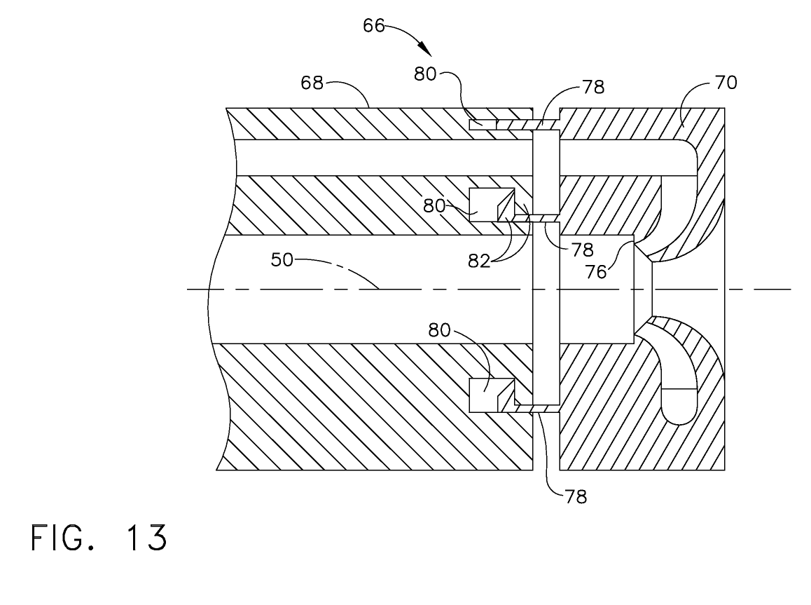

In a first alternate sheath embodiment, as shown in FIG. 13, the sheath 66 includes a mid sheath portion 68 disposed proximal to the manifold 70, and the manifold 70 is manually rotatable about the central longitudinal axis 50 of the sheath 66 with respect to the mid sheath portion 68. In one variation, the manifold 70 and the mid sheath portion 68 are attached by a rotatable (rotatable about the central longitudinal axis 50) tongue 78 and at-least-partially-annular groove 80 arrangement. In one variation, not shown, the distal scope end is angled for improved sideways viewing, the manifold is correspondingly angled, and the rotatable manifold feature allows rotational alignment of the angled manifold with the angled distal scope end.

In one arrangement of the third expression of the first embodiment, as best seen in FIG. 7A, the lumen 30 has an irrigation flow path direction which is substantially parallel to the central longitudinal axis 50 of the sheath 18. In one variation, the manifold 56 has a radiused distal inner wall portion 72 facing the distal lumen end 34. In one example, the radiused distal inner wall portion 72 receives irrigation flow from the distal lumen end 34 and gradually turns such irrigation flow to fill the annular fluid passageway 58 of the manifold 56, such gradual turning reducing pressure losses.

In one configuration of the third expression of the first embodiment, as best seen in FIG. 8, the sheath 18 includes a scope stop 74, and the scope 14 is insertable into the sheath 18 and is translatable within the sheath 18 to abut the scope stop 74 to define the attached sheath 18. In one example, the scope stop 74 is a portion of the manifold 56.

In the first alternate sheath embodiment, as shown in FIG. 13, the sheath 66 includes a mid sheath portion 68 disposed proximal to the manifold 70, and the manifold 70 is manually longitudinally extendable and retractable with respect to the mid sheath portion 68. Here, the scope stop 76 is a portion of the manifold 70. Typically, scopes 14 vary a small amount (such as one inch) in length, and the extendable and retractable manifold 70 allows scopes 14 of varying length to be fully inserted in the sheath 66 against the scope stop 76. In one variation, the manifold 70 and the mid sheath portion 68 are attached by a sliding tongue 78 and groove 80 arrangement and have locking tabs 82 which abut during extension of the manifold 70 with respect to the mid sheath portion 68 to prevent separation of the manifold 70 from the mid sheath portion 68.

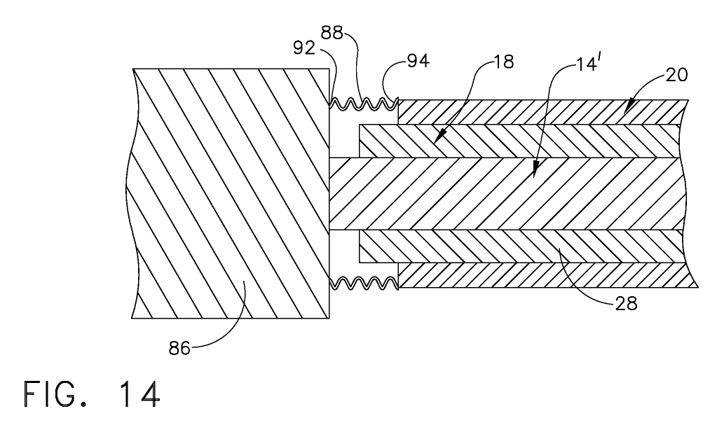

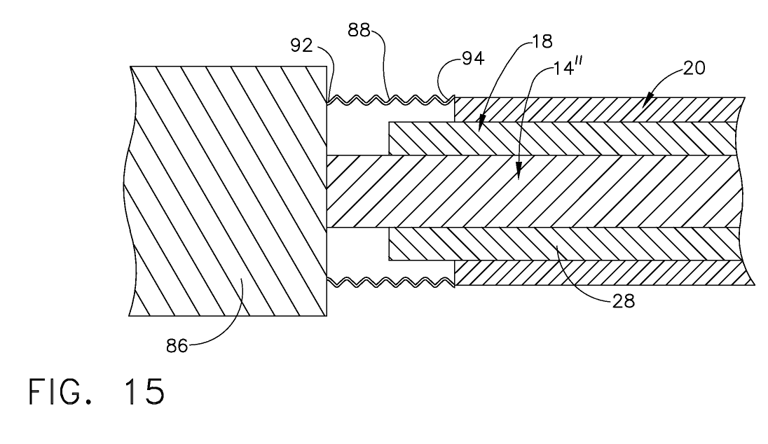

In one employment of the third expression of the first embodiment having the scope stop 74, the distal scope end 12 is the distal scope end 12 of an insertion tube 84 extending from an end of a housing 86 of the scope 14, wherein the sheath 18 is surroundingly attachable to the insertion tube 84 of the scope 14. In this employment, the apparatus 10 also includes, as best shown in FIGS. 1-3 and 14-15, a flexible annular bellows 88 having a central longitudinal axis 90 substantially coaxially aligned with the central longitudinal axis 50 of the sheath 18. The bellows 88 includes a proximal bellows end 92 and includes a distal bellows end 94 which abuts the handpiece 20. The bellows 88 is biased (such as by a spring, not shown) to extend proximally from the handpiece 20 to a fully extended position. The bellows 88 is adapted, for the attached sheath 18, to surround the insertion tube 84 of the scope 14 with the proximal bellows end 92 contacting the end of the housing 86 of the scope 14. This allows a shorter scope 14′ (see FIG. 14) or a longer scope 14″ (see FIG. 15) to be fully inserted against the scope stop 74 (see FIG. 8) without a longitudinal gap between the housing 86 of the scope 14 and the handpiece 20 (see FIG. 3).

In one illustration of the third expression of the first embodiment, the sheath 18 is manually rotatable about the central longitudinal axis 50 of the sheath 18 with respect to the handpiece 20. In one example, the proximal sheath passageway portion 5 has a crescent shape perpendicular to the flow direction. In one variation, not shown, the distal scope end 12 is angled for improved sideways viewing, the manifold 56 is correspondingly angled, and the rotatable sheath feature allows rotational alignment of the angled manifold with the angled distal scope end.

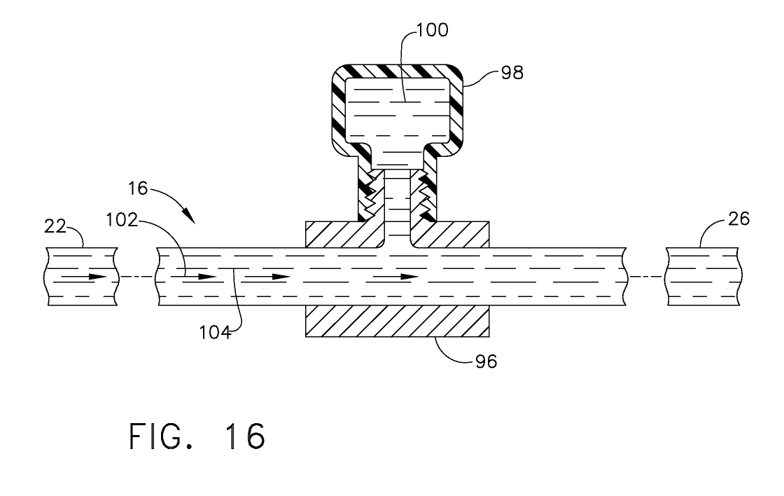

In one extension of the third expression of the first embodiment, as seen in FIG. 16, the first tube 16 includes a fitting 96 which is disposed between the first proximal tube end 22 and the first distal tube end 26. The fitting 96 is adapted to threadably receive a container 98 containing an anti-fogging liquid 100. In one example, the anti-fogging liquid 100 is drawn into the flow 102 of irrigation fluid 104 by the venturi effect.

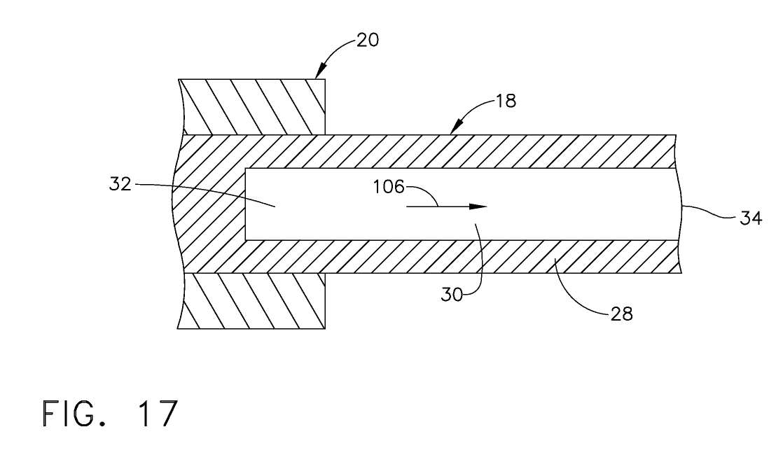

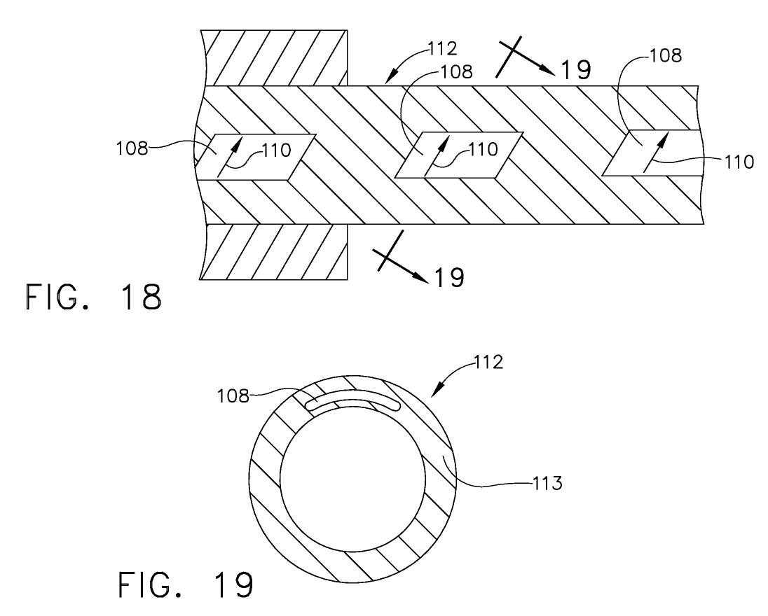

In one employment of the third expression of the first embodiment, as best seen in FIG. 7A and 17, the lumen 30 has a substantially straight proximal-to-distal flow path 106. Flow path 106 is the flow path of the irrigation fluid in lumen 30. It is noted that the vacuum flow path in lumen 30 is the reverse of flow path 106. In an alternate employment, as seen in figures 18 and 19, the lumen 108 has a substantially helical flow path 110. Flow path 110 is the flow path of the irrigation fluid in lumen 108. In one variation (which can be pictured as having the sheath 112 of FIG. 18 substituted for the sheath 18 of FIG. 7A), this allows the distal lumen end 34 to be substantially tangentially aligned with the annular fluid passageway 58 of the manifold 56 which reduces pressure losses.

In one design of the third expression of the first embodiment, the first, second, and third internal flow configurations 1, 2 and 3 of the handpiece 20 are achieved by, as best shown in FIGS. 10-12, having the handpiece 20 include a first valve 114 and a second valve 116. The first valve 114 is operatively disposed between the first distal tube end 26 and the proximal lumen end 32, and the second valve 116 is operatively disposed between the second distal tube end 46 and the proximal lumen end 32. The first valve 114 has a first valve button 118, has a first fluid inlet 120 fluidly connected to the first distal tube end 26 (also seen in FIG. 1), and has a first fluid outlet 122. The second valve 116 has a second valve button 124, has a second fluid inlet 126, and has a second fluid outlet 128 fluidly connected to the second distal tube end 46 (also seen in FIG. 1). The first fluid outlet 122 and the second fluid inlet 126 form the arms of a “Y” whose leg is the distal handpiece passageway portion 4 (seen in FIG. 7A). FIG. 10 shows the first internal flow configuration 1 where the first and second buttons 118 and 124 are biased upward. FIG. 11 shows the second internal flow configuration 2 where the first valve button 118 has been depressed. FIG. 12 shows the third internal flow configuration 3 where the second valve button 124 has been depressed. Arrows 106 indicate the irrigation fluid flow path, and arrows 130 indicated the suction flow path. In one example, the first and second valves 114 and 116 are trumpet valves. In one variation, the saline bag 38 is disposed at a height for sufficient flow of irrigation fluid to quickly impinge the distal scope end 12 once the first valve button 118 is depressed. In one modification, a bladder, not shown, is placed around the saline bag 38 to compress the bag to increase the pressure of the irrigation fluid.

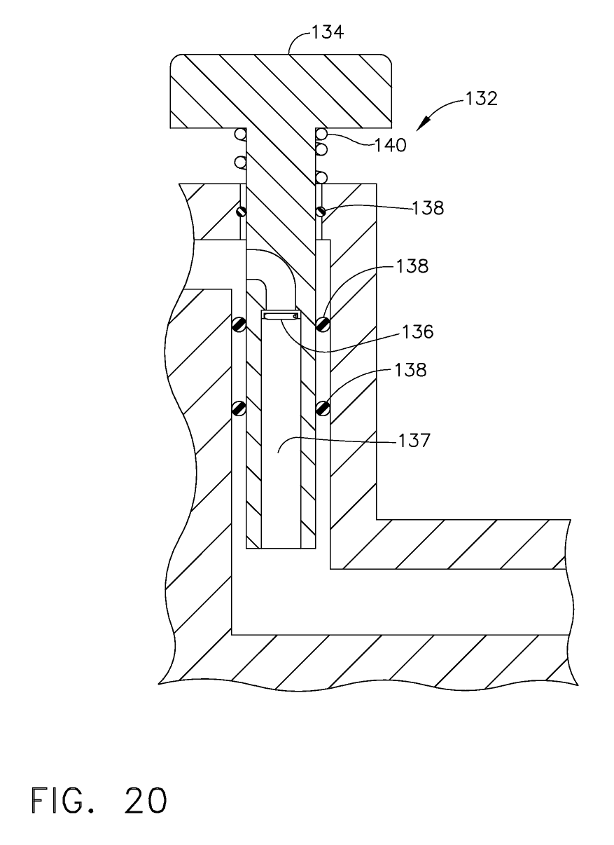

In an alternate embodiment of the first valve, as seen in FIG. 20, the first valve 132 has a first button 134 and is adapted to pump irrigation fluid into the proximal lumen end 32 (also see FIGS. 10 and 7A) when the first button 134 is manually depressed and when the first proximal tube end 22 is fluidly connected to the irrigation fluid source 24. Note the one-way flapper valve 136 (in the button passageway 137 of the first button 134), the O-ring seals 138, and the button return spring 140 of the first valve 132 in FIG. 20. The flapper valve 136 is biased open when the first button 134 is not depressed allowing irrigation fluid to flow downward below the flapper valve 136. When the first button 134 is depressed, the flapper valve 136 is forced shut by the resisting irrigation fluid allowing a pumping action of the irrigation fluid by the downwardly-moving first button 134.

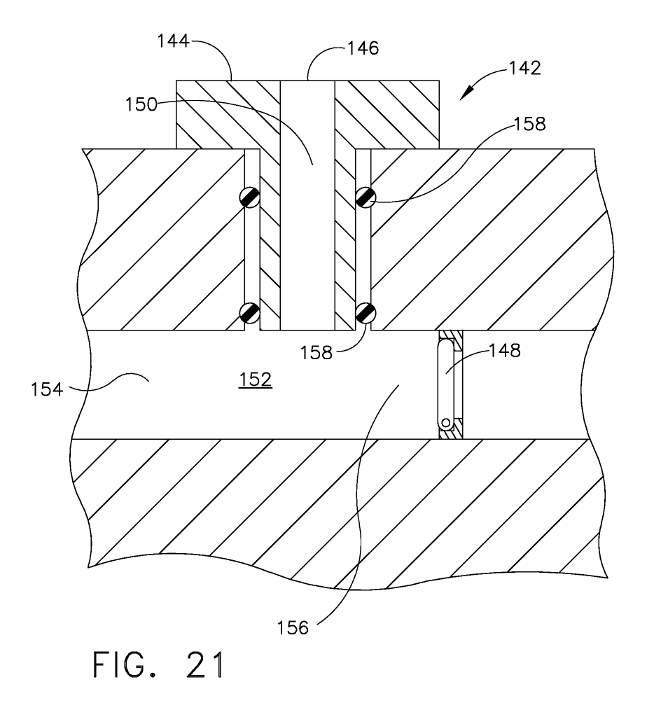

In an alternate embodiment of the second valve, as seen in FIG. 21, the second valve 142 has a second (immovable) valve button 144 with an orifice 146, has a one-way flapper valve 148, and is adapted to suction air from the orifice 146 when the orifice 146 is exposed and is adapted to provide suction to the proximal lumen end 32 when the second valve button 144 is manually covered all when the second proximal tube end 42 is fluidly connected to the vacuum source 44. The orifice 146 is disposed at the top of the second valve button 144 and is in fluid communication with a button passageway 150 of the second valve button 144. The button passageway 150 is in fluid communication with a suction passageway 152 having a first portion 154 extending proximal of the button passageway 150 toward the second distal tube end 46 and having a second portion 156 extending distal of the button passageway 150 toward the proximal lumen end 32. The one-way flapper valve 148 is disposed in the second portion 156 of the suction passageway 152. When the orifice is exposed, the one-way flapper valve 148 is biased shut preventing applying suction to the proximal lumen end 32. When the orifice is covered, the one-way flapper valve 148 is forced open, by the pressure differential, allowing suction to be applied to the proximal lumen end 32. Note the O-ring seals 158.

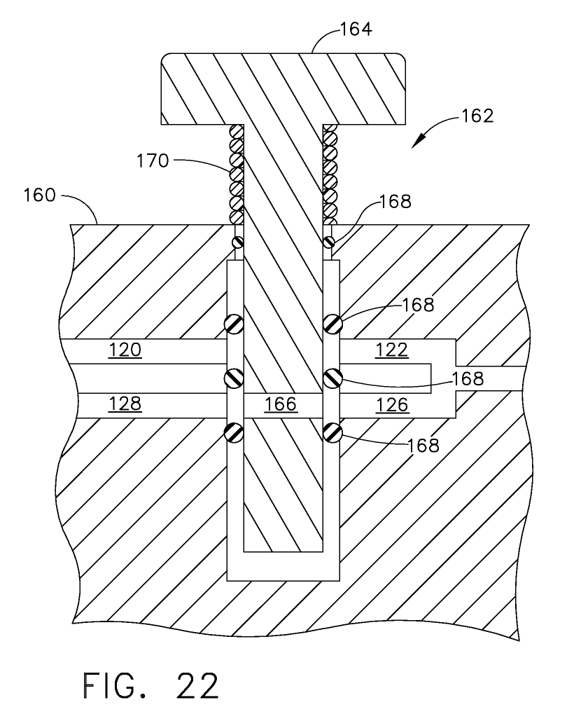

In an alternate embodiment which replaces the first and second valves with a single valve, as seen in FIG. 22, the handpiece 160 includes a single valve 162 having a single valve button 164 having first, second, and third positions, wherein the valve 162 is adapted to provide the first, second, and third internal flow configurations 1, 2, and 3 based correspondingly on the first, second, and third positions of the valve button 164. The valve button 164 has a single transverse button passageway 166. When the valve button 164 is biased upward, the button passageway 166 is not aligned with the first fluid inlet 120 or with the second fluid outlet 128. When the valve button 164 is partially depressed, the button passageway 166 is aligned with the first fluid inlet 120 and the first fluid outlet 122 providing fluid communication between the first fluid inlet 120 and the first fluid outlet 122. When the valve button 164 is fully depressed, the button passageway 166 is aligned with the second fluid inlet 126 and the second fluid outlet 128 providing fluid communication between the second fluid inlet 126 and the second fluid outlet 128. Note the O-ring seals 168, and the button return spring 170 of the valve 162 in FIG. 22.

A second embodiment of the invention is shown in FIGS. 1-12, 14-15, and 17-19, wherein FIGS. 1-12, 14, 15, and 17 illustrate the first embodiment having sheath 18, and wherein FIGS. 18-19 illustrate the lumen portion of a second alternate embodiment of the sheath 112 which replaces the lumen portion of sheath 18 to create the second embodiment. A first expression of the second embodiment is for apparatus 10 for keeping clean a distal scope end 12 of a medical viewing scope 14. The apparatus 10 includes an annular sheath 112 surroundingly attachable to the scope 14, wherein the sheath 112 includes a tubular wall 113 having inside and outside diameters and containing a lumen 108 between the inside and outside diameters, wherein the lumen 108 has proximal and distal lumen ends 32 and 34, wherein the lumen 108 substantially continuously varies in at least one of cross-sectional flow area and irrigation flow path direction, wherein the proximal lumen end 32 is fluidly connectable to at least one of an irrigation fluid source 24 and a vacuum source 44, and wherein the distal scope end 12 is disposed proximate the distal lumen end 34 of the attached sheath 112.

In one variation of the first expression of the second embodiment, the distal scope end 12 is in fluid communication with the distal lumen end 34 of the attached sheath 112. In the same or a different variation, the irrigation fluid flow path 110 has a substantially helical shape with a substantially constant cross-sectional flow area. In one example, the lumen 108 has a substantially crescent shape.

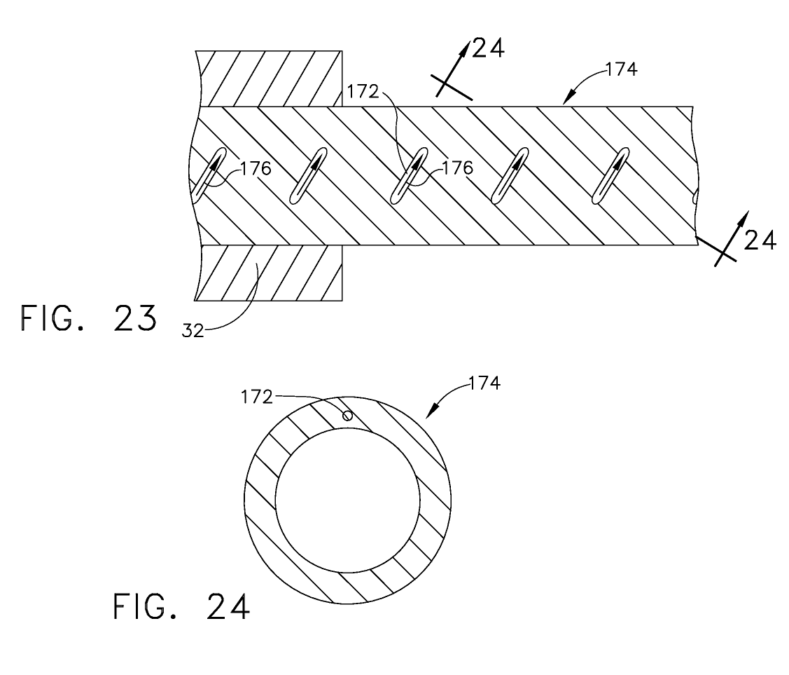

In a third alternate sheath embodiment, as shown in FIGS. 23-24, the lumen 172 of the sheath 174 has a flow path direction 176 (the irrigation fluid flow path direction is shown) which has a substantially helical shape. In this embodiment, the cross-sectional flow area of the lumen 172 is substantially constant and has a substantially circular shape.

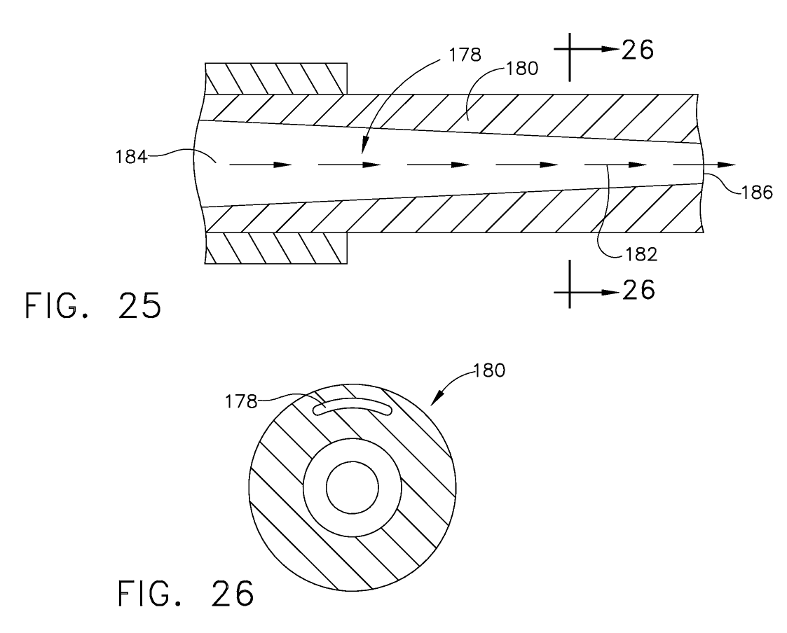

In a fourth alternate sheath embodiment, as shown in FIGS. 25-26, the lumen 178 of the sheath 180 has a flow path direction 182 (the irrigation fluid flow path direction is shown) which is a substantially straight flow path direction. In this embodiment, the cross-sectional flow area of the lumen 178 has a substantially crescent shape which substantially continuously tapers from proximate the proximal lumen end 182 to proximate the distal lumen end 184.

Several benefits and advantages are obtained from one or more of the expressions of embodiments of the invention which provide for keeping clean a distal scope end of a medical viewing scope while the scope remains inserted in a patient. In one example, not removing the scope for cleaning and not reinserting the cleaned scope reduces the time for a laparoscopic procedure. In the same or a different example, not removing the scope for cleaning and not reinserting the cleaned scope keeps the inserted scope aligned with the patient tissue of interest during cleaning so that the physician does not have to take additional time to maneuver the scope to reacquire the patient tissue of interest. In one example of the first and third expressions of the first embodiment, the substantially equal areas reduce flow losses and provide faster response times for irrigation fluid to exit the lumen of the sheath to clean the distal scope end or to clean a magnifying or non-magnifying optional lens (transparent shield) of the sheath which protects the distal scope end.

While the present invention has been illustrated by a description of several expressions, embodiments, and examples, etc. thereof, it is not the intention of the applicants to restrict or limit the spirit and scope of the appended claims to such detail. Numerous other variations, changes, and substitutions will occur to those skilled in the art without departing from the scope of the invention. For instance, the apparatus of the invention has application in robotic assisted surgery taking into account the obvious modifications of such apparatus to be compatible with such a robotic system. It will be understood that the foregoing description is provided by way of example, and that other modifications may occur to those skilled in the art without departing from the scope and spirit of the appended Claims.

What is claimed is:

1. Apparatus for keeping clean a distal scope end of a medical viewing scope, wherein the apparatus comprises:
a) a first tube having a first proximal tube end fluidly connectable to an irrigation fluid source, having a first distal tube end, and having a substantially constant first cross-sectional flow area;
b) an annular sheath surroundingly attachable to the scope, wherein the sheath includes a tubular wall having inside and outside diameters and containing a lumen between the inside and outside diameters, wherein the lumen has proximal and distal lumen ends, wherein the lumen has a substantially constant cross-sectional flow area which is substantially equal in area to the first cross-sectional flow area, and wherein the distal scope end is disposed proximate the distal lumen end of the attached sheath; and
c) a handpiece mounted to the sheath, fluidly connected to the first distal tube end, and in fluid communication with the proximal lumen end, wherein the handpiece is adapted to have a user-selectable first internal flow configuration preventing fluid communication between the first distal tube end and the proximal lumen end and a user-selectable second internal flow configuration allowing fluid communication between the first distal tube end and the proximal lumen end.

2. The apparatus of claim 1, wherein the distal scope end is in fluid communication with the distal lumen end of the attached sheath.

3. The apparatus of claim 1, wherein the first cross-sectional flow area of the first tube has a substantially circular shape, and wherein the cross-sectional flow area of the lumen of the sheath has a substantially crescent shape.

4. The apparatus of claim 3, wherein the scope is a laparoscope, and wherein the sheath is substantially rigid and is insertable into a trocar.

5. The apparatus of claim 4, wherein the irrigation fluid source is an operating-room saline bag, and wherein the first proximal tube end is fluidly connected to the saline bag.

6. Apparatus for keeping clean a distal scope end of a medical viewing scope, wherein the apparatus comprises:
a) a first tube having a first proximal tube end fluidly connectable to an irrigation fluid source and having a first distal tube end;
b) a second tube having a second proximal tube end fluidly connectable to a vacuum source and having a second distal tube end;
c) an annular sheath surroundingly attachable to the scope, wherein the sheath includes a tubular wall having inside and outside diameters and containing a lumen between the inside and outside diameters, wherein the lumen has proximal and distal lumen ends, wherein the lumen has a cross-sectional flow area which has a substantially crescent shape, and wherein the distal scope end is disposed proximate the distal lumen end of the attached sheath; and
d) a handpiece mounted to the sheath, fluidly connected to the first and second distal tube ends, and in fluid communication with the proximal lumen end, wherein the handpiece is adapted to have a user-selectable first internal flow configuration preventing fluid communication between the first distal tube end and the proximal lumen end and between the second distal tube end and the proximal lumen end, a user-selectable second internal flow configuration allowing fluid communication between the first distal tube end and the proximal lumen end but not between the second distal tube end and the proximal lumen end, and a user-selectable third internal flow configuration allowing fluid communication between the second distal tube end and the proximal lumen end but not between the first distal tube end and the proximal lumen end.

7. The apparatus of claim 6, wherein the distal scope end is in fluid communication with the distal lumen end of the attached sheath.

8. The apparatus of claim 6, wherein the first tube has a substantially constant first cross-sectional flow area, wherein the second tube has a substantially constant second cross-sectional flow area which is substantially equal in area to the first cross-sectional flow area of the first tube, wherein the lumen has a substantially constant cross-sectional flow area, wherein the first and second cross-sectional flow areas each have a substantially circular shape, and wherein the cross-sectional flow area of the lumen is substantially equal in area to the first cross-sectional flow area of the first tube.

9. The apparatus of claim 8, wherein the scope is a laparoscope, and wherein the sheath is substantially rigid and is insertable into a trocar.

10. The apparatus of claim 9, wherein the irrigation fluid source is an operating-room saline bag, wherein the first proximal tube end is fluidly connected to the saline bag, wherein the vacuum source is an operating-room suction canister, and wherein the second proximal tube end is fluidly connected to the suction canister.

11. Apparatus for keeping clean a distal scope end of a medical viewing scope, wherein the apparatus comprises:
a) a first tube having a first proximal tube end fluidly connectable to an irrigation fluid source, having a first distal tube end, and having a substantially constant first cross-sectional flow area;
b) a second tube having a second proximal tube end fluidly connectable to a vacuum source and having a second distal tube end;
c) an annular sheath having a central longitudinal axis, surroundingly attachable to the scope, and insertable into a patient, wherein the sheath includes a tubular wall having inside and outside diameters and containing a lumen between the inside and outside diameters, wherein the lumen has proximal and distal lumen ends, wherein the lumen has a substantially constant cross-sectional flow area which is substantially equal in area to the first cross-sectional flow area of the first tube, wherein the sheath includes a distal sheath end portion defining a manifold, wherein the manifold has an annular fluid passageway which has a volume and which is in fluid communication with the distal lumen end, wherein the manifold has a plurality of spaced apart nozzle passageways which together have a total volume, which are in fluid communication with the annular fluid passageway, and which point proximal of the annular fluid passageway, wherein the volume of the annular fluid passageway is greater than the total volume of the nozzle passageways, and wherein the distal scope end is disposed proximate the nozzle passageways of the attached sheath; and
d) a handpiece mounted to the sheath, fluidly connected to the first and second distal tube ends, and in fluid communication with the proximal lumen end, wherein the handpiece is adapted to have a user-selectable first internal flow configuration preventing fluid communication between the first distal tube end and the proximal lumen end and between the second distal tube end and the proximal lumen end, a user-selectable second internal flow configuration allowing fluid communication between the first distal tube end and the proximal lumen end but not between the second distal tube end and the proximal lumen end, and a user-selectable third internal flow configuration allowing fluid communication between the second distal tube end and the proximal lumen end but not between the first distal tube end and the proximal lumen end.

12. The apparatus of claim 11, wherein the distal scope end is in fluid communication with the nozzle passageways.

13. The apparatus of claim 11, wherein the second tube has a substantially constant second cross-sectional flow area which is substantially equal in area to the first cross-sectional flow area of the first tube, wherein the first and second cross-sectional flow areas each have a substantially circular shape, and wherein the cross-sectional flow area of the lumen of the sheath has a substantially crescent shape.

14. The apparatus of claim 13, wherein the crescent shape is substantially equal in shape to an end view of substantially ninety degrees of a circularly annular right cylinder.

15. The apparatus of claim 13, wherein the scope is a laparoscope, and wherein the sheath is substantially rigid and is insertable into a trocar.

16. The apparatus of claim 15, wherein the irrigation fluid source is an operating-room saline bag, wherein the first proximal tube end is fluidly connected to the saline bag, wherein the vacuum source is an operating-room suction canister, and wherein the second proximal tube end is fluidly connected to the suction canister.

17. The apparatus of claim 11, wherein each nozzle passageway has a proximal passageway end and a distal passageway end, and wherein each nozzle passageway is tapered from the corresponding proximal passageway end to the corresponding distal passageway end.

18. The apparatus of claim 11, wherein each nozzle passageway has a proximal passageway end and a distal passageway end, and wherein the distal passageway end of the attached sheath has a portion abutting the distal scope end and a portion spaced apart from the distal scope end.

19. The apparatus of claim 11, wherein the sheath includes a mid sheath portion disposed proximal to the manifold, and wherein the manifold is manually rotatable about the central longitudinal axis of the sheath with respect to the mid sheath portion.

20. The apparatus of claim 11, wherein the lumen has an irrigation flow path direction which is substantially parallel to the central longitudinal axis of the sheath, and wherein the manifold has a radiused distal inner wall portion facing the distal lumen end.

21. The apparatus of claim 11, wherein the sheath includes a scope stop, and wherein the scope is insertable into the sheath and is translatable within the sheath to abut the scope stop to define the attached sheath.

22. The apparatus of claim 21, wherein the sheath includes a mid sheath portion disposed proximal to the manifold, and wherein the manifold is manually longitudinally extendable and retractable with respect to the mid sheath portion.

23. The apparatus of claim 22, wherein the manifold and the mid sheath portion are attached by a sliding annular tongue and annular groove arrangement and have locking tabs which abut during extension of the manifold with respect to the mid sheath portion to prevent separation of the manifold from the mid sheath portion.

24. The apparatus of claim 21, wherein the distal scope end is the distal scope end of an insertion tube extending from an end of a housing of the scope, wherein the sheath is surroundingly attachable to the insertion tube of the scope, and also including a flexible annular bellows having a central longitudinal axis substantially coaxially aligned with the central longitudinal axis of the sheath, wherein the bellows includes a proximal bellows end and includes a distal bellows end which abuts the handpiece, wherein the bellows is biased to extend proximally from the handpiece to a fully extended position, and wherein the bellows is adapted, for the attached sheath, to surround the insertion tube of the scope with the proximal bellows end contacting the end of the housing of the scope.

25. The apparatus of claim 11, wherein the sheath is manually rotatable about the central longitudinal axis of the sheath with respect to the handpiece.

26. The apparatus of claim 11, wherein the first tube includes a fitting which is disposed between the first proximal tube end and the first distal tube end and which is adapted to threadably receive a container containing an anti-fogging liquid.

27. The apparatus of claim 11, wherein the lumen has a substantially straight flow path.

28. The apparatus of claim 11, wherein the lumen has a substantially helical flow path, and wherein the distal lumen end is substantially tangentially aligned with the annular fluid passageway of the manifold.

29. The apparatus of claim 11, wherein the handpiece includes a first valve operatively disposed between the first distal tube end and the proximal lumen end and a second valve operatively disposed between the second distal tube end and the proximal lumen end.

30. The apparatus of claim 29, wherein the first valve has a first valve button and is adapted to pump irrigation fluid into the proximal lumen end when the first valve button is manually depressed and when the first proximal tube end is fluidly connected to the irrigation fluid source.

31. The apparatus of claim 29, wherein the second valve has a second valve button with an orifice and a one-way flapper valve and is adapted to suction air from the orifice when the orifice is exposed and is adapted to provide suction to the proximal lumen end when the second valve button is manually covered all when the second proximal tube end is fluidly connected to the vacuum source.

32. The apparatus of claim 11, wherein the handpiece includes a single valve having a single valve button having first, second, and third positions, wherein the valve is adapted to provide the first, second, and third internal flow configurations based correspondingly on the first, second, and third positions of the valve button.

33. Apparatus for keeping clean a distal scope end of a medical viewing scope, wherein the apparatus comprises an annular sheath surroundingly attachable to the scope, wherein the sheath includes a tubular wall having inside and outside diameters and containing a lumen between the inside and outside diameters, wherein the lumen has proximal and distal lumen ends, wherein the lumen substantially continuously varies in at least one of cross-sectional flow area and irrigation flow path direction, wherein the proximal lumen end is fluidly connectable to at least one of an irrigation fluid source and a vacuum source, and wherein the distal scope end is disposed proximate the distal lumen end of the attached sheath.

34. The apparatus of claim 33, wherein the distal scope end is in fluid communication with the distal lumen end of the attached sheath.

35. The apparatus of claim 33, wherein the flow path direction has a substantially helical shape.

36. The apparatus of claim 33, wherein the path direction is a substantially straight irrigation flow path direction, and wherein the cross-sectional flow area of the lumen has a substantially crescent shape which substantially continuously tapers from proximate the proximal lumen end to proximate the distal lumen end.
